(19) United States
(12) Patent Application Publication
(10) Pub. No.: US 2022/0257392 A1
(43) Pub. Date: Aug. 18, 2022

(54) Devices and Methods for Treating Aneurysms and Other Vascular Conditions

(71) Applicant: LionRock Endovascular, Inc., Irvine, CA (US)

(72) Inventor: Yuk Hang Wong, New Territories (HK)

(73) Assignee: LionRock Endovascular, Inc., Irvine, CA (US)

(21) Appl. No.: 17/674,643

(22) Filed: Feb. 17, 2022

Related U.S. Application Data
(60) Provisional application No. 63/150,088, filed on Feb. 17, 2021.

(51) Int. Cl. A61F 2/86; A61F 2/00

(52) U.S. Cl. CPC A61F 2/86; A61F 2002/823; A61F 2/0077

(57) ABSTRACT

An embolization device has a metal stent structure, and a plurality of fiber strands attached to the outer surface of the metal stent structure. An intermediate transition structure can also surround the metal stent structure, with the plurality of fiber strands is attached to an outer surface of the intermediate transition structure. In use, the embolization device is first delivered to the location of an aneurysm, and then a stent-graft is introduced into the lumen of the embolization device and expanded for deployment inside the lumen of the embolization device.

### BACKGROUND OF THE INVENTION

### 1. Field of the Invention

The present invention relates to devices and methods for treating aneurysms and other vascular conditions, and in particular, to an embolization device for use with a stent-graft.

### 2. Description of the Prior Art

An aneurysm is a weak section of an artery wall. Pressure from inside the artery causes the weakened area to bulge out beyond the normal size/dimension of the blood vessel. Aneurysms can occur anywhere in the arterial circulation of the human body, such as in the brain and the aortic, among other locations.

The aorta is the largest blood vessel in the body. It delivers oxygenated blood from the heart to the rest of the body. An aortic aneurysm is a bulging, weakened area in the wall of the aorta. Over time, the blood vessel balloons and is at risk for rupture or separation (dissection). This can cause life-threatening bleeding and potentially death. Aneurysms occur most often in the portion of the aorta that runs through the abdomen (abdominal aortic aneurysm). An abdominal aortic aneurysm is also called AAA or triple A.

A thoracic aortic aneurysm refers to an aneurysm at the part of the aorta that runs through the chest.

Once formed, an aneurysm will gradually increase in size and become progressively weaker. When left untreated, the aneurysm may rupture, or vessel dissection may happen, usually causing rapid fatal hemorrhaging.

Treatment for an abdominal aneurysm may include open surgical repair or endovascular aortic aneurysm repair (EVAR) using a stent-graft device. Compared with surgical repair, the EVAR procedure is less invasive, and carries with it a reduced mortality rate along with shorter stays in the hospital and the intensive care unit.

In recent years, there have been a number of stent-grafts and AAA endoprostheses that have been approved and which are commercially available. While EVAR provides benefit for the patients who are eligible for the procedure, there still some difficulties/disadvantages associated with current EVAR technologies that must be overcome.

For example, according to VQI data, the 5-year mortality of the patients treated with EVAR have inferior outcomes compared to open surgery. The key difference between EVAR and open surgery are as follows. Open surgery involves the complete closure of the flow lumen as well as removal of the mural thrombus. However, EVAR procedures cannot remove the mural thrombus and the flow lumen is expected to thrombose. It is believed that the thrombus in the sac of the aneurysm could be an active mass contributing to the inferior long term clinical outcome of the patients.

Thus, it is desirable to provide an improved device/technology to address above-mentioned drawbacks and other related issues experienced by current EVAR devices and procedures.

### SUMMARY OF THE DISCLOSURE

In order to accomplish the objects of the present invention, there is provided an embolization device (hereinafter “device”) with fibers attached thereto, which can be deployed with a stent-g raft during an EVAR procedure to induce thrombosis in the aneurysm sac.

The embolization device has a metal stent structure, and a plurality of fiber strands attached to the outer surface of the metal stent structure. An intermediate transition structure can also surround the metal stent structure, with the plurality of fiber strands is attached to an outer surface of the intermediate transition structure. In use, the embolization device is first delivered to the location of an aneurysm, and then a stent-graft is introduced into the lumen of the embolization device and expanded for deployment inside the lumen of the embolization device.

The potential benefits of the device of the present invention include, but are not limited to: 1) induce aneurysm thrombosis to reduce or eliminate Type-2 endoleak; 2) induce platelet aggregation and fibrin network formation to enhance the stability of the thrombus. The stable fibrin rich thrombus is expected to enhance the aneurysm shrinkage.

Other advantages of the device of the present invention include, but are not limited to:
1) A big mass of the thrombotic fibers can be introduced into the sac at once.
2) It is much more user-friendly and cost-effective than the deployment of multiple embolization coils/materials or devices.
3) Without adding artifacts for CT/MRI.
4) It is compatible to a majority of existing AAA devices in the market without requiring a learning curve for the clinician.

### BRIEF DESCRIPTION OF THE DRAWINGS

FIG. 1A is a two-dimensional view of the stent structure 100a of a first embodiment made from sheet material.

FIG. 1B is a two-dimensional view of the stent structure 100b of a second embodiment made from sheet material, which has a different cell pattern at its proximal end 120b.

FIG. 2A is a perspective view of the stent 100a of FIG. 1A. Note the split/gap 110a along the longitudinal direction for diameter adjustment once the stent is deployed to accommodate the dimension of any branches of the stent-graft. This split/gap can be a negative distance in the case where the sheet material is overlapped.

FIG. 2B is a perspective view of the stent 100b of FIG. 1B. Note the split/gap 110b along the longitudinal direction for diameter adjustment once the stent is deployed to accommodate the dimension of any branches of the stent-graft. This split/gap can be a negative distance in the case where the sheet material is overlapped.

FIG. 3 is a two-dimensional view of the stent structure 100c of a third embodiment made from sheet material.

FIG. 4 is a perspective view of the stent 100c of FIG. 3. Note the split/gap 110c along the longitudinal direction for diameter adjustment once the stent is deployed to accommodate the dimension of any branches of the stent-graft. This split/gap can be a negative distance in the case where the sheet material is overlapped.

FIG. 5A is a two-dimensional view of the fiber and the thin sheet of material that forms the intermediate transition structure 200.

FIG. 5B is a magnified view of FIG. 5A.

FIG. 6 is a perspective view of a fiber sheet 200 that forms the intermediate transition structure 200, with fiber strands 300 secured on the outer surface of the intermediate transition structure 200. Note that gaps or splits 210 can also be provided along the longitudinal direction, and the two edges of the intermediate transition structure can also be overlapped.

FIG. 7 is a perspective view of a device of the present invention having the intermediate transition structure 200 and fiber strands 300 of FIG. 6, with the stent structure 100a of FIG. 1A secured to the inner surface of the intermediate transition structure.

FIG. 8 is an end view of the device of FIG. 7 taken from the proximal end thereof.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The embolization devices of the present invention can be embodied in the manner disclosed in the following embodiments. The following can be considered to be the basic principles of the present invention.

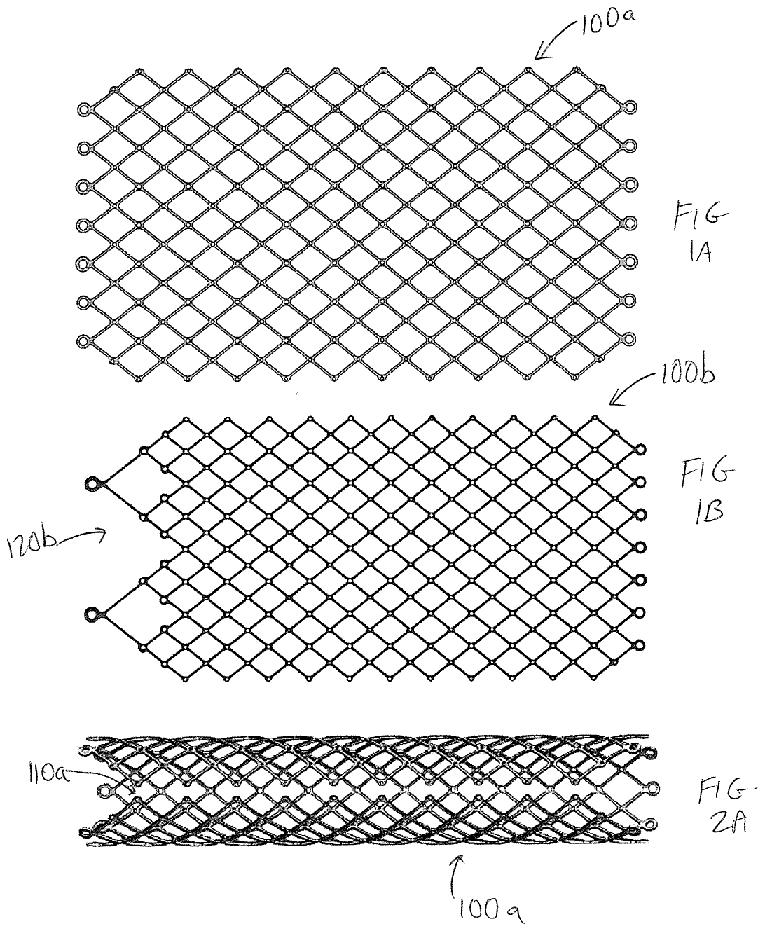

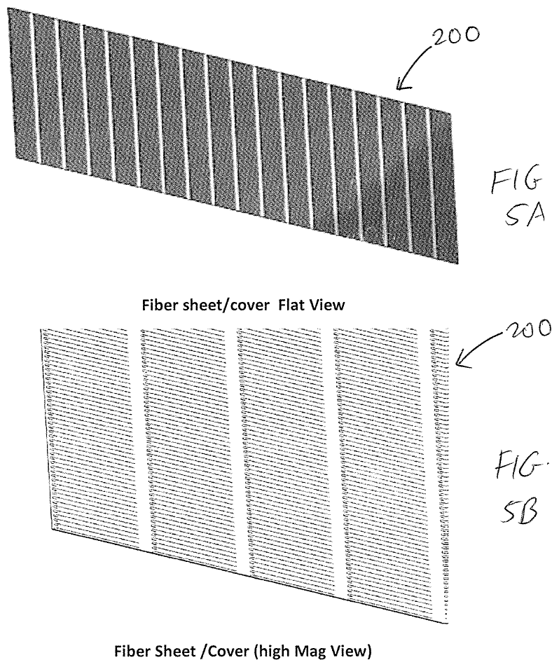

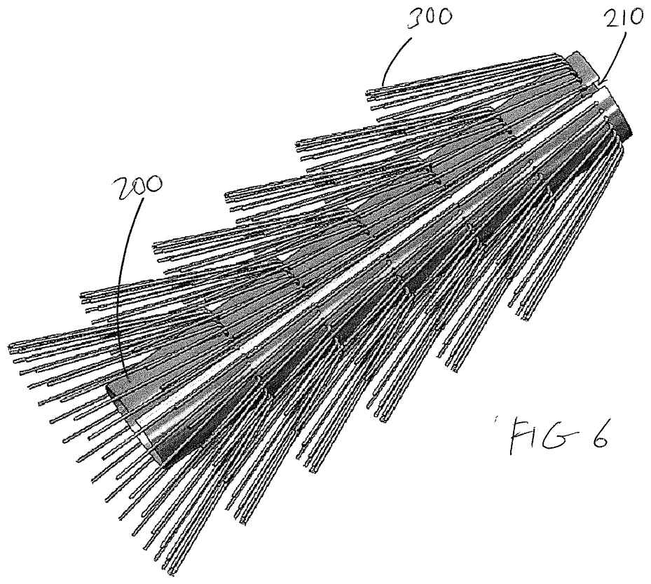

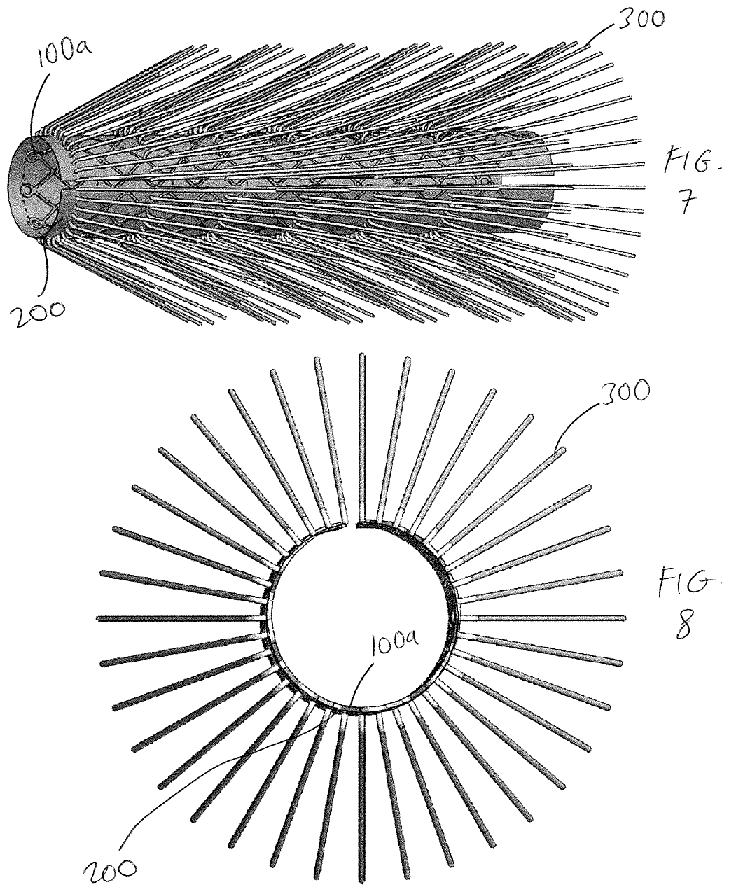

As best shown in FIG. 7, each embolization device according to the present invention has an underlying stent structure 100a (FIG. 1A), 100b (FIG. 1B) or 100c (FIG. 3), an optional intermediate transition structure 200 surrounding the stent structure, and fiber strands 300 secured to either the stent structure or the intermediate transition structure. In use, the embolization device is first delivered to the location of an aneurysm, and expanded and anchored to a healthy portion of the underlying blood vessel (e.g., aorta). Next, a stent-graft device is introduced into the lumen of the embolization device and expanded for deployment inside the lumen of the embolization device. Thus, when the EVAR procedure is completed, the embolization device surrounds the stent-graft.

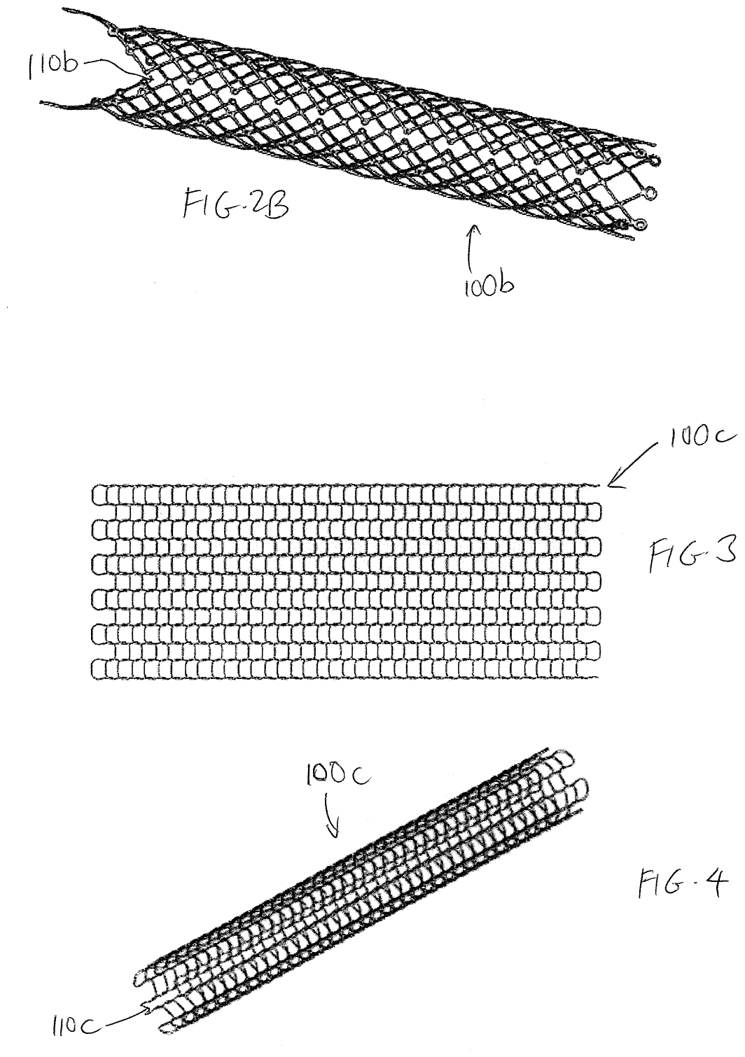

In one embodiment, the stent structure 100a, 100b or 100c can be rolled or foldable (see FIG. 2a, 2b or 4), and made from a sheet material (or tubular material), by laser cut, EDM, chemical machining, electrochemical machining, or other similar means. Polymer fibers (e.g., Dacron™ fibers) can be attached onto the stent structure directly, or be attached onto the stent structure through a fiber sheet acting as an intermediate transition structure. The attachment methods include, but are not limited to, mechanical attachment, adhesion, and thermal bonding. The intermediate transition structure includes, but is not limited to, knitted wire, ribbon structure, polymer textures, polymer cloth, polymer sheets, etc. The sheet or tubular material can be made from Nitinol material, DFT Nitinol material, Co—Cr alloys, Ta alloys, stainless steel, and other biocompatible polymer materials. The stent structure can be either self-expandable or balloon expandable. This foldable stent structure may or may not need a shape setting process to define its expanded dimension. If needed, drug(s) can also be integrated into the fibers to promote the healing of the aneurysm sac.

In another embodiment, the stent structure 100a, 100b or 100c can be a hand or machine fabricated woven wire stent structure, which can be folded or rolled (see FIG. 2a, 2b or 4) to form various diameters. One device can be made from either a single wire or multiple wires. The wire stent structure can have either open or closed cell structure. Polymer fibers (e.g., Dacron™ fibers) can be attached onto the stent structure directly, or be attached onto the stent structure through a fiber sheet acting as an intermediate transition structure. The attachment methods include, but are not limited to, mechanical attachment, adhesion, and thermal bonding. The intermediate transition structure includes, but is not limited to, knitted wire, ribbon structure, polymer textures, polymer cloth, polymer sheets, etc. The sheet or tubular material can be made from Nitinol material, DFT Nitinol material, Co—Cr alloys, Ta alloys, stainless steel, and other biocompatible polymer materials. The stent structure can be either self-expandable or balloon expandable. This foldable stent structure may or may not need a shape setting process to define its expanded dimension. If needed, drug(s) can also be integrated into the fibers to promote the healing of the aneurysm sac.

Both stent structures disclosed above can either have a uniform diameter throughout the entire length, or a flared structure at one end or both ends. One example is that if the stent structure has a flared distal end (relative to the delivery system), then the flared distal end can provide two benefits. First, the flared distal end can reduce the possibility for the embolization device to extend into the entrance of any branch or leg of the stent-graft upon deployment. Second, the flared distal end can be pushed up after partial deployment (without entering any portion of the stent-graft) to increase the fibers around the main body of the stent-graft, in the proximal lumen/sac of the aneurysm.

The embolization devices of the present invention can be mounted and delivered through an 8-14Fr Over-The-Wire (OTW) delivery system during the EVAR procedure. The OTW delivery system can be made from polymer materials, and may include: (1) a handle to operate the device; (2) one or more through-lumens on the inner core to allow a guidewire to extend through; (3) an outer sheath to constrain and release the device; (4) an atraumatic distal tip; and (5) markers/marker bands for positioning purposes.

The majority of the stent-grafts used in EVAR procedures have a modular design, so the embolization devices of the present invention can be easily adapted for use with any of these available stent-grafts. The embolization device can be delivered to the target location via the pre-existing guidewire for the stent-graft, then deployed by unsheathing the outer sheath of its delivery system. Once deployed at the target location, as the embolization device has a foldable structure, it will be compatible with, or accommodate, any diameters of the branches of the stent-graft and can be further expanded by the branches (if needed) to achieve optimal interface with between the embolization device and the branches of the stent-graft.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A device, comprising:
a metal stent structure; and
a plurality of fiber strands attached to the outer surface of the metal stent structure.

2. The device of claim 1, further including an intermediate transition structure surrounding the metal stent structure, and wherein the plurality of fiber strands is attached to an outer surface of the intermediate transition structure.
